(19) United States
(12) Patent Application Publication
(10) Pub. No.: US 2003/0021819 A1
(43) Pub. Date: Jan. 30, 2003

(54) Microbial and odor control using amorphous calcium silicate impregnated with sodium chlorite

(75) Inventors: Neeraj Khanna, Norman, OK (US); Robert J. Vahlberg, Norman, OK (US); Theodore D. Head, Manhattan, IL (US); Bryan D. Lowery, Oklahoma City, OK (US)

Correspondence Address: Crowe & Dunlevy, 1800 Mid-America Tower, 20 North Broadway, Oklahoma City, OK, 73102-8273, US

(73) Assignee: Bio-Cide International, Inc., Norman, OK

(21) Appl. No.: 10/126,603

(22) Filed: Apr. 18, 2002

Related U.S. Application Data
(63) Continuation-in-part of application No. 09/670,067, filed on Sep. 26, 2000.
Application No. 09/670,067 is a continuation-in-part of application No. 09/251,051, filed on Feb. 18, 1999, now Pat. No. 6,132,748.
(60) Provisional application No. 60/075,289, filed on Feb. 19, 1998.
(60) Provisional application No. 60/284,532, filed on Apr. 18, 2001.

(30) Foreign Application Priority Data
Feb. 18, 2000 (US) PCT/US00/04289

(51) Int. Cl. A01N 25/00; A61K 33/24; A61K 33/14

(52) U.S. Cl. 424/405; 424/617; 424/677

(57) ABSTRACT

The present invention is directed to the a composition and method for microbial and odor control. In one embodiment, the method includes mixing amorphous calcium silicate impregnated with a chlorite salt to form a reactant, combining the reactant with an activator to form the composition, and applying the composition to the treatment area. The present invention also includes the preparation of a product usable as a disinfectant and deodorizer, wherein the product includes a mixture of an amorphous and a chlorite salt and wherein the product is packaged as a tablet, permeable sachet, or a permeable patch attachable to a plastic bag. The present invention may also be applied using several methods to reduce the spoilage of produce. The present invention further includes a method for producing chlorine dioxide in accordance with a step-function release profile.

### RELATED PATENTS AND APPLICATIONS

The present application claims the benefit of U.S. Provisional Patent Application Serial No. 60/284,532, of the same title, filed Apr. 18, 2001. The present application is also a continuation-in-part of U.S. patent application Ser. No. 09/670,067, entitled PREVENTION OF SPOILAGE OF PRODUCE USING ACIDIFIED EXPANDED AMORPHOUS ALUMINUM SILICATE IMPREGNATED WITH CHLORITE, filed Sep. 26, 2000. U.S. patent application Ser. No. 09/670,067 is a continuation of the application that issued as U.S. Pat. No. 6,132,748, entitled METHOD FOR PRODUCING CHLORINE DIOXIDE USING ACIDIFIED EXPANDED AMORPHOUS ALUMINUM SILICATE IMPREGNATED WITH CHLORITE, filed Feb. 18, 1999. The application that issued as U.S. Pat. No. 6,132,748 claims the benefit of U.S. Provisional Patent Application Serial No. 60/075,289, of the same title, which was filed Feb. 19, 1998. Each of the related applications listed above are hereby incorporated by reference.

### FIELD OF INVENTION

The present invention relates to the production of chlorine dioxide using amorphous silicates impregnated with chlorite salts.

### BACKGROUND OF INVENTION

Chlorine dioxide (ClO₂) is a superior oxidizing agent that is capable of penetrating the cell walls, membranes and cytoplasm of mold spores, bacteria and other microbiological contaminants at low concentrations. Because of its biocidal efficacy, chlorine dioxide is commonly used as a disinfectant or fumigant in a number of applications and environments. Recently, chlorine dioxide has been used to disinfect food products during the packaging process.

The incorporation of chlorine dioxide or sodium chlorite in food packaging has prompted studies to determine whether residual levels of such preservatives result in a significant genetic or carcinogenic hazard to humans. Meier et al. studied the effect of subchronic and acute oral administration of chlorine, chlorine dioxide, sodium chlorite, sodium chlorate and related substances on the induction of chromosomal aberrations and sperm head abnormalities in mice. Only the highly reactive hypochlorite resulted in a weak positive effect for mutagenic potential. The other compounds, including chlorine dioxide and sodium chlorite, failed to induce any chromosomal aberrations or increased numbers of micronuclei in the bone marrow of mice. Richardson et al. reported that an extensive study of the reaction of chlorine dioxide with water borne organics by the Environmental Protection Agency confirmed this observation.

Similarly, ClO₂ has also been used as a deodorant. Japanese Patent No. 63/296,758 issued Kokai and assigned to the NOK Corporation describes a deodorant created by impregnating micro porous beads with an aqueous solution of stabilized chlorine dioxide and wrapped in non-woven cloth. Japanese Patent No. 57/168,977 issued to Encler Business describes a deodorant product that contains chlorine dioxide incorporated with a calcium silicate molded product containing about 0.01-0.2 weight percentage of iron (Fe₂O₃) and having a petal-like crystal structure.

Gels which generate chlorine dioxide for use as topical applications for disinfection are disclosed by Kenyon, et. al., Am. J. Vet. Res., 45(5), 1101 (1986). Chlorine dioxide generating gels are generally formed by mixing a gel containing suspended sodium chlorite with a gel containing lactic acid immediately prior to use to avoid premature chlorine dioxide release. Chlorine dioxide releasing gels have also been used in food preservation.

Encapsulation processes have also been used in preparing sources of chlorine dioxide. Canadian Patent No. 959,238 describes generation of chlorine dioxide by separately encapsulating sodium chlorite and lactic acid in polyvinyl alcohol and mixing the capsules with water to produce chlorine dioxide.

Tice, et al., U.S. Pat. No. 4,585,482, describe gradual hydrolysis of alternating poly(vinyl methyl ether-maleic anhydride) or poly(lactic-glycolic acid) to generate acid which can release chlorine dioxide from sodium chlorite. A polyalcohol humectant and water are encapsulated with the polyanhydride or polyacid in a nylon coating. After sodium chlorite is diffused into the capsule through the nylon wall, an impermeable polystyrene layer is coacervated around the nylon capsule. Solvents are required for reaction and application of the capsules. The capsules can be coated onto surfaces to release chlorine dioxide. Although the capsules are said to provide biocidal action for several days to months, chlorine dioxide release begins immediately after the capsules are prepared. The batchwise process used to prepare the capsules also involves numerous chemical reactions and physical processes, some of which involve environmental disposal problems.

Despite the many prior art applications of ClO₂, there is a continued need for a composite that can be easily activated to initiate chlorine dioxide release in use. A composition that is composed of only FDA approved substances, or those generally recognized as safe (GRAS), is particularly needed for food packaging and other applications where the substances are ingested or contacted by humans.

### SUMMARY OF THE INVENTION

In one aspect, the present invention is directed to the a composition and method for microbial and odor control. The method includes mixing amorphous calcium silicate impregnated with a chlorite salt to form a reactant, combining the reactant with an activator to form the composition, and applying the composition to the treatment area. The present invention also includes the preparation of a product usable as a disinfectant and deodorizer, wherein the product includes a mixture of an amorphous and a chlorite salt and wherein the product is packaged as a tablet, permeable sachet, or a permeable patch attachable to a plastic bag.

In another aspect, the present invention includes a method for reducing the spoilage of produce by applying amorphous calcium silicate impregnated with a chlorite salt and thereafter stimulating the release of chlorine dioxide by contacting the reactant with an activator. An alternative method includes applying an activator to the produce, wherein the activator includes amorphous calcium silicate impregnated with an acid, and thereafter applying a chlorite salt solution to the activated produce to stimulate the release of chlorine dioxide.

The present invention further includes a method for producing chlorine dioxide in accordance with a step-function release profile. The inventive method includes mixing amorphous silicate with a chlorite salt to produce a reactant, combining the reactant with an activator to form a composition, adding a desiccant to the composition and thereafter exposing the desiccant and composition to moisture.

The advantages and features of the present invention will be apparent from the following description when read in conjunction with the accompanying drawings and appended claims.

### BRIEF DESCRIPTION OF THE DRAWINGS

FIG. 1 shows ClO₂ release from accelerated release amorphous silicate; optimized release amorphous silicate; and extended release amorphous silicate.

FIG. 2 shows a step-function ClO₂ profile with an initial delay phase.

FIG. 3 shows a patch constructed in accordance with the present invention.

### DETAILED DESCRIPTION OF THE INVENTION

The present invention provides a composition and method for producing a source of chlorine dioxide (ClO₂). The composition includes the combination of a reactant and an activator. The reactant serves as the source of chlorine dioxide and preferably includes a silicate that is mixed with a chlorite salt. In the preferred embodiment, the chlorite salt is sodium chlorite. It will be noted, however, that other counter cations of the chlorite salt can be also used, such as potassium, calcium, barium or any other suitable cation as would be recognized by those skilled in the art.

In some cases, it is desirable to prepare the reactant by soaking the amorphous silicate with an aqueous solution of from about 0.01% to about 50% chlorite anion by weight. For example, soaking can be achieved by spraying a 5% solution of sodium chlorite, while agitating the amorphous silicate mechanically. The soaked amorphous silicate is dried at a temperature of about 120° C. for about two hours and sealed in an air-tight container or with a desiccator to prevent premature moisture absorption. A presently preferred source of sodium chlorite solution is commercially available from BioCide International, Inc. of Norman, Okla. under the ProOxine® trademark.

The reactant produces chlorine dioxide in response to exposure to the activator. While most amorphous silicates have some inherent acidity, the inherent acidity is low enough that without the use of an activator, the reactant would produce chlorine dioxide in small amounts over an extended period. A more rapid release of ClO₂ may be desired for deodorization and/or sterilization.

In a preferred embodiment, the activator includes an acid. If the acid is powdered, the acid can be combined with a suitable silicate to facilitate mixing with the reactant. Suitable powdered acids include citric, succinic, salicylic, oxalic acid, sulfamic, GRAS acids and powdered alum.

Alternatively, the activator can be prepared by soaking a suitable silicate with from about 0.01% to about 50% of liquid acid. The liquid acid can be sprayed on the silicate and allowed to dry. The liquid acids that can be used to prepare the activator include, without limitation, phosphoric, hydrochloric, sulphuric, nitric, acetic, tartaric, glycolic, mandelic, malic, maleic, aspartic, lactic, propanoic or other structurally similar acids. The concentration of the soaking solution can range from about 0.01 M to saturated, depending on the desired potency of the activator.

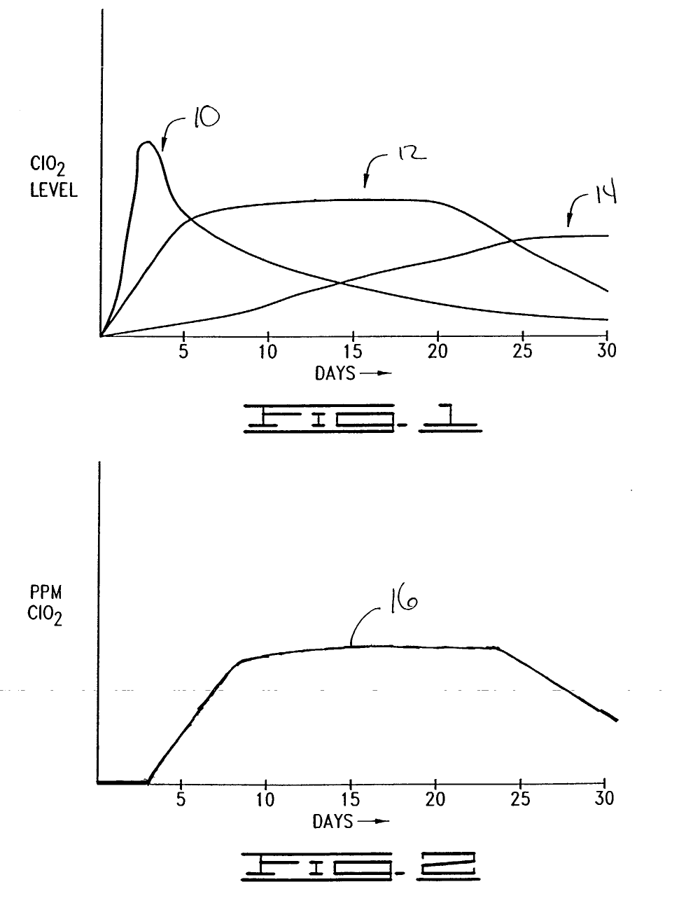

Exposing the reactant to the activator in specific ratios produces different chlorine dioxide release profiles, as shown in FIG. 1. In general, the release rates of chlorine dioxide are expressed as either accelerated 10, moderated 12 or extended 14. To make a product that will provide accelerated release, a greater amount of activator is added. To make a product that will provide moderated release, less activator is added. Finally, to make a product that will provide an extended release, even lower amounts of activator are used, or the activator is totally eliminated. Some of these methods are discussed in U.S. Pat. No. 6, 132,748 issued Oct. 17, 2000 to Khanna et al. (Khanna '748) referenced above.

Silicates that can be used in the reactant and activator include amorphous silicates, such as calcium silicate, magnesium silicate, aluminum silicates, silicic acids, silicate gels, other precipitated silicas, various varieties of clays, and mixtures thereof. Many amorphous silicates are naturally occurring, while others must be synthesized. For example, calcium silicates are produced through the reaction of lime with diatomaceous earth. The word “amorphous” is used to reflect the absence of a definite crystalline structure.

In a preferred embodiment, the silicate used is amorphous calcium silicate. Amorphous calcium silicates are ideal for use in chlorine dioxide releasing systems for several reasons. Amorphous calcium silicates have varied particle size and shape, high surface area and low bulk density. Generally, the particle size in amorphous calcium silicate is between 10 to 1000 microns. This variable particle size creates a heterogeneous environment that aids in the sustained release of chlorine dioxide in the present invention.

Calcium silicates also conform to the Federal Drug Administration's (FDA) CODEX (database) requirements of safe additives. Calcium silicates have a GRAS (generally regarded as safe) status that allow their use in food, beverage, and pharmaceutical products as evidenced by 21 C.F.R. 573.260, which states that, “calcium silicate, including synthetic calcium silicate, may be safely used as an anti-caking agent in animal feed, provided that the amount of calcium silicate does not exceed 2 percent.” An amorphous calcium silicate that is suitable for the present invention is manufactured by World Mineral Inc. of Santa Barbra, Calif., under the Micro-Cel® trademark. It will be noted that another form of amorphous silicate that behaves similarly is magnesium silicate.

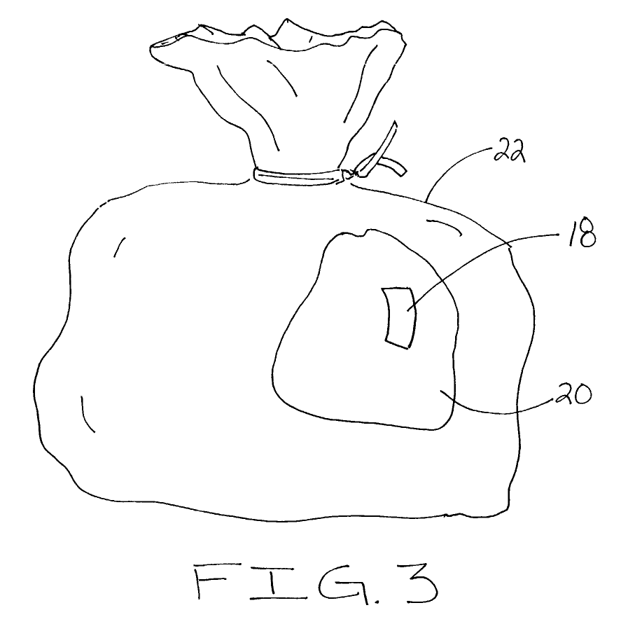

Amorphous calcium silicates also have among the highest absorptive capacity of known silicates. The exceptionally high absorption capacity minimizes the amount of inert carrier needed to convert liquids to dry powders. This high absorptivity also aids in the design of various release profiles for the chlorine dioxide gas. For example, the use of amorphous calcium silicates in the reactant creates variably delayed “step-function” release profiles when exposed to moisture, such as profile 16 shown in FIG. 2.

Preceding a threshold level of hydration, moisture is used to satisfy hydration sites within the amorphous calcium silicate. Beyond the threshold of hydration, the moisture reacts with the chlorite salt to produce chlorine dioxide. The delay in release can be further enhanced by adding a desiccant, such as anhydrous calcium chloride.

Although the present invention is not limited by a particular mechanism, a likely mechanism of ClO₂ release may be explained as follows. Water molecules in moisture provide the medium that facilitates the interaction of chlorite ions with protons present in the activator and/or amorphous silicate. The chlorite ions probably react with the protons according to the following equation:

5ClO₂⁻+4H⁺→4ClO₂(g)+Cl⁻+2H₂O

One advantage of the present invention is the moisture-induced solid phase release of ClO₂ that creates an antimicrobial and deodorizing atmosphere at the site of application. As suggested above, in low ambient moisture environments, moisture can be introduced to accelerate ClO₂ production; however, normal humidity may supply the necessary moisture. The amorphous nature of the matrix provides a much longer time-range for sustained release of ClO₂ as compared to a matrix that is homogeneous in nature. The extended time-range results from the existence of a range of channel-sizes (˜10 to 1000 Å) in the amorphous substance that extends the kinetic time scale for the penetration of the water molecules.

The present invention can be used for the microbial control of dry or semi dry goods such as produce, cosmetics, medical devices, paper fabric, plastics, fertilizers and other agricultural items. This present invention is also suited for use in odor control, since ClO₂ has been shown to exhibit excellent deodorizing properties.

A variety of applications and packaging techniques can be adopted to apply the present invention. For example, the inventive composition can be applied directly to a treatment area in a premixed form, with or without packaging. Alternatively, the reactant and activator can be applied separately to the treatment area, through use of spraying or fogging devices. Several preferred methods of application are described below.

Due to its absorptive characteristics, amorphous calcium silicates allow a much higher liquid content with low friability. Amorphous calcium silicates also exhibit excellent compressibility and recompressibility characteristics that enable the use of these silicates in formed or shaped products. For example, in a preferred embodiment, the inventive composition is manufactured as in a tablet form. As used herein, the term “tablet” will be used to denote all sizes and shapes of formed products, including pills, sticks and pucks. Disinfectant and deodorant tablets can be made by mixing sodium chlorite solutions with amorphous calcium silicates. Tablets serve as a convenient method of packaging and can be used to remove odor in areas such as toilets and closets.

The inventive composition can also be packaged in moisture permeable sachets or patches. Generally, sachets are portable containers that are capable of being moved to multiple treatment areas. In contrast, as shown in FIG. 3, a patch 18 is generally adhered to the walls or inner lining 20 of a larger container, such as a garbage bag 22. Such sachets and patches are suitable for attachment or placement in plastic bags, garbage cans or other confined treatment areas.

Suitable sachets and patches may be constructed of spin-bonded olefins, such as Tyvek®, or non-woven polyethylene materials. A patch can also be manufactured by extruding the reactant in layered plastic. Extruded patches can include an exterior adhesive that enables placement of the patch to the treatment area. Because they function much like common adhesive tape, such extruded patches are particularly well suited for placement in a variety of treatment areas.

The permeability of the sachet and patch enables moisture to diffuse into the sachet and patch while retaining the inventive composition. Preferably, the inventive composition is sealed from moisture until the production of ClO₂ is desired. In an alternative embodiment, a push/pull bottle can be used to store and activate the dry, chemically impregnated ingredients on as-need basis.

The inventive composition can also be used in combination with “anti-block” products that are used to coat plastic films. Anti-block products prevent the fusion or sticking of proximate or adjacent surfaces. The incorporation of the inventive composition with an anti-block product is particularly useful when used to coat the interior surfaces of plastic bags.

In another embodiment, the release of chlorine dioxide gas can be triggered by exposing the chlorite-impregnated amorphous silicate to a volatile acid such as acetic acid. Such an application will be particularly useful in a low humidity environment or in situations where the use of activator is prohibited due to pH requirements of the composition. In such an application the volatile acid can also be mixed with the moisture.

In another application, the reactant is placed in direct contact with produce and thereafter exposed to a suitable activator, through a misting or fogging apparatus. Alternatively, both reactant and activator can be applied to produce and thereafter fogged with moisture to accelerate the release of chlorine dioxide.

An example of such use is for potatoes, also referred to as tubers, after harvest. Potatoes are typically stored for up to 10 months, and one of the biggest challenges for long-term potato storage is the prevention of spoilage from bacteria and fungi. Common potato spoilage can include soft rot (caused by Erwinia carotovora), dry rot (caused by Fusarium sambucinum), and silver scurf (caused by Helminthosporium solani) which can be intensified by a fungus such as Phytophthora infestans which can result in the collapse of potato piles in storage facilities.

It is well known that chlorine dioxide is quite effective in controlling all the above mentioned spoilage organisms. The efficacy data for Purogene, a chlorine dioxide product manufactured by Bio-Cide International, against the organisms Phytophthora infestans and Helminthosporium solani are listed in the tables provided below in Example 9. Additional data including other organisms is available in Olsen et al., BUL 825, College of Agriculture, University of Idaho, 1999.

Currently, solutions of chlorine dioxide are being used in many storage facilities to prevent spoilage of potatoes. However, in some situations where the harvested potatoes are too wet when placed into storage, this practice has not worked well. Spraying chlorine dioxide solution on already wet potatoes leads to extreme wetness which is believed to propagate late blight. It is well known that the germination of P. infestans zoospores is facilitated in the presence of excess water, and the benefit of the biocidal powder of chlorine dioxide is offset. In such situations a non-aqueous source of chlorine dioxide is desired.

The above described use of chlorine dioxide generation via amorphous silicate works well on stored potatoes because this method does not require excess water. Additionally, since amorphous silicate is water absorbent, it helps dry the wet tubers and facilitates disease management. Furthermore, potatoes are typically stored under high moisture, a condition that encourages the production of chlorine dioxide from the reactant as discussed above.

In a particularly preferred embodiment, the reactant is prepared by mixing 1 part sodium chlorite with 20 parts of amorphous calcium silicate and applied to tubers while moving along a conveyer belt of a piling machine. For potatoes that have high degree of infection, the release of chlorine dioxide from the reactant can be accelerated by adding an acidic activator.

This reactant can also be applied, with or without the activator, on previously piled potatoes in storage. In powder form, the reactant can be blown into storage houses using an air stream. Since most storage houses are designed to maintain high humidity (90 to 95%), the reactant releases chlorine dioxide when exposed to moisture. Also, since amorphous silicate is generally mixed with agricultural soil as a porosity enhancing diluent, the application of antimicrobial chlorite-impregnated amorphous silicate mixture is particularly useful on seed potatoes that require additional protection after planting in soil. In such applications, the reactant produces ClO₂ upon contact with moisture present in the soil.

Currently, it is common to apply chlorine dioxide solutions in storage houses through humidification systems. Concentrated chlorine dioxide solutions are added in the humidification waters and the gas is carried by the vapor phase into potatoes piles. A major drawback of this approach is that, due to the labile nature of the chlorine dioxide gas, most of the chlorine dioxide gas reacts with the tubers located on the surface of the pile and very little penetrates deeper.

An alternative embodiment of the present invention provides an improved method of distributing chlorine dioxide in piled produce, such as potatoes. In this embodiment, the potatoes are sprayed with a powdered activator (1 part citric acid: 5 part amorphous silicate) and then piled as usual. Subsequently, whenever the release of chlorine dioxide is required, the piled potatoes are fogged with a sodium chlorite solution. Since sodium chlorite is much less reactive than the chlorine dioxide gas, the sodium chlorite solution penetrates deeper into the pile. After penetration, the chlorite ions contact the acidic activator powder, thereby producing chlorine dioxide at the surface of individual tubers. Since chlorite ion is the limiting reagent, the piles can be fogged several times to produce biocidal chlorine dioxide gas. In this embodiment, the amorphous silicate can be added to the activator to prevent the absorption of acid by the tuber and to provide protonation sites that serve as a medium for a sustained reaction.

Other suitable applications for the present invention will be readily recognized by those skilled in the art, all of which are within the spirit and scope of the present invention.

### EXAMPLE 1

The composition used in this experiment contained 100 grams of dehydrated calcium silicate, 12.8 grams of sodium chlorite and 20 grams of citric acid. The sodium chlorite used in this experiment is 80% pure and commercially available from Vulcan Chemicals of Birmingham, Ala. Other sources and purities of sodium chlorite may be also used. The sodium chlorite and citric acid were mixed individually with calcium silicate prior to mixing them together. 6.5 grams of this composition were placed on a 4-inch diameter petri dish which was then placed in 1 gallon jars that were maintained between 80% to 95% relative humidity and between 20° C. and 25° C. The jars were made of poly (ethylene-terephthalate) material commonly known as PET. The jars were kept in the ambient lab environment and the inside humidity and temperature was monitored. The humidity inside these jars was maintained by spraying calculated amounts of water into the jars. The humidity was monitored with a hygrometer manufactured by Radio Shack (model 63-867A).

To measure the concentration of ClO₂ released from the amorphous silicate product, the lid was closed for a specified period of time and the ClO₂ levels were measured with a chlorine dioxide monitoring device that is commercially available from Mil-Ram Technologies, Inc., San Jose, Calif. under the Tox-Array 1000 trademark. The chlorine dioxide monitoring device was calibrated to measure a rang of concentrations from 0.1 to 20 ppm of ClO₂. For each measurement the sample was drawn from the top of the jar by opening the lid slightly and allowing the insertion of the sample suction tube into the jar. The suction tube was directly connected to the monitoring device.

The release profile is reported in Table 1. There was no chlorine dioxide produced for the first 40 hours. This observation is attributed to the fact that the initial moisture absorption is utilized for satisfying the hydration sites in the calcium silicate lattice and is not available for catalyzing the reaction of chlorite to chlorine dioxide. Thus there is a delay period (as shown in FIG. 2) that can be manipulated by varying the degree of hydration in the calcium silicate raw material.

TABLE 1

| Hours | ppm of ClO₂ |
|---|---|
| 0 | 0 |
| 8 | 0 |
| 16 | 0 |
| 24 | 0 |
| 32 | 0 |
| 40 | 0 |
| 48 | 1 |
| 56 | 1.3 |
| 64 | 2.2 |
| 72 | 2.1 |
| 80 | 3.3 |
| 88 | 3.2 |
| 96 | 4.0 |
| 104 | 6.3 |

### EXAMPLE 2

The composition in this experiment contained 100 grams of dehydrated magnesium silicate, 12 grams of sodium chlorite and 20 grams of citric acid. Sodium chlorite and citric acid were mixed individually with magnesium silicate prior to mixing them together. 11 Grams of this composition was introduced in the a PET gallon jar. The conditions and the measurement techniques were the same as described in Example 1. The release profile is reported in Table 2. Unlike the release profile of chlorine dioxide with calcium silicate, chlorine dioxide production started within the first eight hours of the exposure to humidity.

TABLE 2

| Hours | ppm of ClO₂ |
|---|---|
| 0 | 0 |
| 8 | 0.4 |
| 16 | 0.6 |
| 24 | 1.2 |
| 32 | 2 |
| 40 | 2.1 |
| 48 | 1.9 |
| 56 | 2.1 |
| 64 | 2.4 |
| 72 | 3.0 |
| 80 | 3.1 |
| 88 | 2.9 |
| 96 | 2.8 |
| 104 | 2.9 |

### EXAMPLE 3

This experiment focused on the odor abatement properties of chlorine dioxide that is released from chlorite-impregnated amorphous silicates when exposed to moisture. The four odor-causing compounds that were tested are thiophene, 2-mercaptoethanol , trimethylamine and isovaleric acid. These compounds were purchased from the Aldrich Chemical Company of St. Louis, Mo. Thiophene and 2-mercaptoethanol form the basis for rotten, sulfureous odors, such as the odors exhibited by rotten eggs or human waste. Trimethylamine forms the basis of rotten seafood odors and isovelaric acid forms the basis of rancid dairy products.

Two sets of four pieces of 2in.×2in. filter paper were soaked with 10 μl of four different odor causing compounds and dried for 2 minutes. The filter paper was placed in eight 13-gallon garbage cans such that the first set of four cans, each with a different compound, was used as a control. An amount of the powdered composition from Example 1 was introduced into the second set. This composition contained calcium silicate, sodium chlorite, and citric acid in the specified ratio. An amount of 5 grams of powder was placed in a petri dish and the petri dish was placed at the bottom of each can. An amount of 2 μl of water was misted into all the cans (including the controls) to generate humidity for ClO₂ release. After 8 hours, the petri dishes containing the ClO₂ generating powder were removed from the cans and the cans were evaluated for odor control by a panel of five individuals.

The panel concluded that there was a significant reduction in the odor in the cans that were exposed to the chlorite-impregnated calcium silicate, as compared with the respective controls. The thiophene and the 2-mercaptoethanol odors were completely eliminated. The trimethylamine and isovelaric acid odors were reduced by approximately 80% and 50%, respectively.

### EXAMPLE 4

In the following examples 4 through 8, the amorphous silicate used was expanded amorphous aluminum silicate (EAAS). It was obtained from two different sources: 1) Paradigm International, Inc., Calif. and 2) Aldrich Chemical Company, Milwaukee, Wis. These materials are subsequently P1 and P2, respectively. The density of P2 is much higher than that of P1.

In this experiment 230 milliliters of 0.6M hydrochloric acid was sprayed on each of the 230 g of P1 and P2. These substances were sprayed with a generic spray bottle, with thorough stirring between every few sprays. The acidified amorphous silicate was allowed to bake at 250° C. for one hour. The amorphous silicate turned slightly brown in color, which was possibly due to oxidation of Fe^{2 +} to Fe^{3 +}.

The amorphous silicate product was packaged and used in a 50 cc wide-mouth bottle made of high density polyethylene (HDPE). The cap on the bottle had a push-pull mechanism for sealing or allowing the diffusion of air with the environment via an opening of 0.8 cm diameter. The ClO₂ gas that is generated by the product is discharged into the environment through this opening.

Two bottles of each P1 and P2 were kept in three different locations for trials of odor removal. The results are reported in Tables 3 and 4 below. Samples A and B were kept in a toilet facility (100 sq. ft.), samples B and C were kept in the laboratory (1,600 sq. ft.), and sample E and F were kept in an office (1,500 sq. ft.).

TABLE 3
Product made from P1
Free ClO₂ (ppm)

| | Incu- | | | | | | |
|---|---|---|---|---|---|---|---|
| | bation | Sample | Sample | Sample | Sample | Sample | Sample |
| Days | Time | A | B | C | D | E | F |
| 0 | 15 min | 6.4 | 6.5 | 6.5 | 6.6 | 6.4 | 6.5 |
| 1 | 15 min | 0.4 | 0.7 | 1.3 | 1.2 | 1.1 | 1.8 |
| 5 | 4 hours | 5.0 | 4.8 | 5.2 | 5.3 | 4.8 | 4.7 |
| 6 | 4 hours | 2.1 | 2.0 | 2.1 | 2.1 | 1.0 | 2.1 |
| 7 | 4 hours | 2.2 | 2.8 | 2.5 | 2.3 | 1.3 | 2.8 |
| 8 | 4 hours | 1.3 | 1.5 | 1.3 | 1.0 | 0.5 | 1.3 |
| 11 | 4 hours | 1.2 | 2.5 | 1.5 | 1.0 | 0.3 | 1.2 |
| 12 | 4 hours | 1.5 | 2.4 | 1.7 | 1.8 | 0.5 | 1.3 |
| 13 | 4 hours | 1.2 | 1.9 | 1.4 | 1.4 | 0.6 | 1.1 |
| 14 | 4 hours | 3.1 | 3.2 | 2.1 | 2.3 | 0.4 | 1.4 |
| 15 | 4 hours | 2.3 | 2.8 | 2.2 | 2.3 | 0.7 | 0.8 |
| 18 | 4 hours | 1.6 | 1.7 | 0.5 | 1.1 | 0.7 | 0.1 |
| 19 | 4 hours | 2.1 | 2.0 | 1.1 | 1.8 | 0.9 | 0.2 |
| 21 | 4 hours | 2.0 | 2.4 | 1.0 | 2.0 | 1.2 | 0.5 |
| 22 | 4 hours | 1.8 | 1.9 | 1.0 | 1.8 | 0.8 | 0.3 |

TABLE 4
Product made from P2
Free ClO₂ (ppm)

| | Incu- | | | | | | |
|---|---|---|---|---|---|---|---|
| | bation | Sample | Sample | Sample | Sample | Sample | Sample |
| Days | Time | A | B | C | D | E | F |
| 0 | 15 min | 9.8 | 9.9 | 9.7 | 9.9 | 9.8 | 9.8 |
| 1 | 15 min | 2.7 | 3.5 | 3.3 | 3.2 | 2.6 | 3.1 |
| 4 | 15 min | 0.3 | 0.9 | 0.8 | 0.8 | 1.0 | 0.9 |
| 5 | 1 hour | 4.3 | 4.0 | 3.0 | 3.3 | 2.3 | 3.0 |
| 6 | 1 hour | 0.9 | 2.1 | 1.5 | 1.4 | 1.1 | 1.5 |
| 7 | 1 hour | 0.9 | 2.0 | 1.4 | 1.3 | 0.5 | 1.2 |
| 8 | 1 hour | 0.5 | 1.4 | 1.4 | 0.5 | 0.4 | 0.7 |
| 11 | 1 hour | 0.4 | 1.2 | 0.9 | 1.0 | 0.3 | 0.7 |
| 12 | 1 hour | 0.8 | 1.5 | 1.2 | 1.1 | 0.6 | 1.1 |
| 13 | 1 hour | 1.1 | 1.5 | 0.8 | 0.7 | 0.3 | 1.1 |
| 14 | 1 hour | 3.3 | 3.2 | 2.0 | 1.8 | 0.9 | 1.6 |
| 15 | 1 hour | 4.1 | 3.1 | 1.6 | 1.4 | 0.4 | 0.6 |
| 18 | 1 hour | 3.8 | 3.5 | 0.7 | 1.3 | 0.9 | 0.7 |
| 19 | 1 hour | 5.1 | 4.9 | 1.5 | 2.1 | 2.3 | 1.7 |
| 21 | 1 hour | 3.9 | 4.8 | 3.0 | 3.5 | 3.1 | 1.1 |
| 22 | 1 hour | 2.5 | 3.6 | 2.1 | 2.5 | 1.9 | 0.9 |

### EXAMPLE 5

In this example, the effect the odor elimination effect of the P1 composition described in Example 4 was studied on mercaptoethanol. 25 μl of 2-mercaptoethanol (Aldrich) was tested by two PET jars of the type described above. In the first jar, a bottle containing 5 g of P1 was placed. The second control jar had no product placed in it. Lids sealed both jars. After 12 hours, the product bottle was taken out and the jars aired for 30 minutes. Subsequently, the jars were tested for mercaptan odor by 5 different individuals. None of them could detect any odor in the first jar, whereas the control-jar had a strong odor of mercaptan. The mechanism for the odor removal is believed to be the oxidation of the mercaptan by ClO₂.

### EXAMPLE 6

In this experiment the present invention is very effective in removing onion odors. 25 g of chopped white onions were stored in two PET jars overnight. The onions were removed the next day and the bottle with P1 product was placed in one of the jars. After 12 hours, the jars were inspected for odor by 5 different individuals. The odor was eliminated from the jar that was treated with the P1 product.

### EXAMPLE 7

In this experiment four samples, each containing 5 g of P1, were treated with 0.5 milliliters, 1 milliliter, 3 milliliters and 5 milliliters of 0.6 M HCl. Similarly, four examples each containing 10 g of P2, were treated with 0.5 milliliters, 1 milliliter, 3 milliliters and 5 milliliters of 0.6 M HCl. These samples were allowed to air dry on the laboratory bench, and after one week. 0.5 g NaClO₂ was added. These samples were packaged in the 50 cc bottles described in a prior Example and the ClO₂ levels were monitored in the similar manner as earlier mentioned. In these cases the characteristics of ClO₂ release matched that of accelerated release as shown in the FIG. 1. The results are presented in the following tables.

TABLE 5
Product made from P1
Free ClO₂ (ppm)

| | Incu- | 0.5 milli- | 1 milli- | 3 milli- | 5 milli- |
|---|---|---|---|---|---|
| | bation | liters | liters | liters | liters |
| Days | Time | Acid | Acid | Acid | Acid |
| 0 | 1 hour | 4.5 | 6.8 | 7.3 | 4.2 |
| 1 | 1 hour | 0.4 | — | — | — |
| 2 | 1 hour | 0.0 | 1.8 | — | — |
| 3 | 1 hour | — | — | 0.0 | — |
| 5 | 1 hour | — | 5.6 | — | — |
| 6 | 1 hour | — | 0.0 | — | 0.0 |

TABLE 6
Product made from P2
Free ClO₂ (ppm)

| | Incu- | 0.5 milli- | 1 milli- | 3 milli- | 5 milli- |
|---|---|---|---|---|---|
| | bation | liters | liters | liters | liters |
| Days | Time | Acid | Acid | Acid | Acid |
| 0 | 1 hour | 7.6 | 1.0 | 11.2 | 10.6 |
| 1 | 1 hour | 2.4 | 9.3 | — | — |
| 2 | 1 hour | — | 3.4 | — | — |
| 3 | 1 hour | — | — | 1.2 | — |
| 4 | 1 hour | — | — | — | — |
| 5 | 1 hour | 0.0 | 0.4 | — | — |
| 6 | 1 hour | — | 0.1 | — | — |

### EXAMPLE 8

In this example, NaClO₂is mixed with P1 and P2 that were not treated with any acid. The ratio of mixing was 0.5 g NaClO_{2: 5} g P1 and 0.5 g NaClO_{2: 10} g P2. In these cases, the characteristics of ClO₂ release matched the extended release profiles shown in FIG. 1. The ClO₂ level released from the 50cc bottle (described in Example 1) were below the detection limit of the Tox-Array monitoring device. However, when bulk amounts of both P1 and P2 formulations were left in the PET jars for approximately 1 ½ months, ˜10 ppm and ˜6 ppm of ClO₂ was detected, respectively.

### EXAMPLE 9

Chlorine dioxide solutions can limit the growth of Phytophthora infestans and Helminthosporium solani as shown in the following results:

TABLE 7
Number of living sporangia of Phytophthora infestans
after incubation at 7° C. for 2 hrs:

| | | | | | Average |
|---|---|---|---|---|---|
| Purogene (ppm) | 1 | 2 | 3 | 4 | sporangia/mL |
| 100 | 0 | 0 | 0 | 0 | 0 |
| 50 | 0 | 0 | 0 | 0 | 0 |
| 25 | 0 | 0 | 0 | 0 | 0 |
| 12.5 | 0 | 0 | 0 | 0 | 0 |
| 6.25 | 0 | 0 | 0 | 0 | 0 |
| 3.25 | 4,000 | 2,000 | 0,0 | 0,0 | 3,000 |
| 0.0 | 9,000 | 12,000 | 9,000 | 11000 | 10,250 |

TABLE 8
Percent of germinated zoospores of Phytophthora infestans after
incubation at 20° C. for 24 and 48 hrs:

| Purogene (ppm) | 24 hrs (%) | 48 hrs (%) |
|---|---|---|
| 100 | 0 | 0 |
| 50 | 0 | 0 |
| 25 | 0 | 0 |
| 12.5 | 0 | 0 |
| 6.25 | 0 | 0 |
| 3.12 | 59 | 48 |
| 0.0 | 78 | 75 |

TABLE 9
Percent germinated spores of Helminthosporium solani after
incubation for 48 hrs at 20° C.:

| Purogene (ppm) | Germinated Spores (%) |
|---|---|
| 100 | 0.0 |
| 50 | 0.0 |
| 25 | 51.0 |
| 12.5 | 82.0 |
| 6.25 | 88.0 |
| 3.25 | 86.0 |
| 0.0 | 91.0 |

It is clear that the present invention is well adapted to carry out the objects and to attain the ends and advantages mentioned as well as those inherent therein. While presently preferred embodiments of the invention have been described in varying detail for purposes of the disclosure, it will be understood that numerous changes may be made which will readily suggest themselves to those skilled in the art and which are encompassed within the spirit of the invention disclosed and as defined in the above text, accompanying drawings and appended claims.

The following references as well as those separately cited above are incorporated in pertinent part by reference herein for the reasons cited:

1) Greenwood, N. N., Eamshaw, A. In Chemistry of the Elements;

Pergamon Press: New York, 1989, pp399-416;

2) Perlite Institute Inc., 88 New Drop Plaza, Staten Island, N.Y. 10306-2994;

3) Masschelein, W. J. In Chlorine Dioxide, Chemistry and Environmental Impact of Oxychlorine Compounds; Ann Arbor Science: Ann Arbor, 1979;

4) Wellinghoff, et. al., U.S. Pat. No. 5,695,814;

5) Tice, et al., U.S. Pat. No. 4,585,482;

6) Meier, et al., Environ. Mutagenesis, 7, 201 (1985);

7) Richardson, et al., Environ. Sci. Technol., 28, 592 (1994); and

8) Kenyon et al., Am. J. Vet. Res., 45(5), 1101 (1986).

It is claimed:

1. A method for disinfecting and deodorizing a treatment area with a chlorine dioxide producing composition, the method comprising:
mixing amorphous calcium silicate with a chlorite salt to form a reactant;
combining the reactant with an activator to form the composition, wherein the activator includes an acid; and
applying the composition to the treatment area.

2. The method of claim 1, wherein the chlorite salt is sodium chlorite.

3. The method of claim 1, wherein a desiccant is added to the amorphous calcium silicate to control the release profile of the chlorine dioxide.

4. The method of claim 1, wherein the activator further includes amorphous calcium silicate.

5. The method of claim 1, wherein the acid is a GRAS acid.

6. The method of claim 1, further comprising the step of forming the composition into a tablet prior to the step of applying the composition to the treatment area.

7. The method of claim 1, further comprising the step of mixing the composition with an anti-block prior to the step of applying the composition to the treatment area.

8. The method of claim 7, wherein the mixture of the composition and anti-block is used to coat plastic bags.

9. The method of claim 1, further comprising the step of placing the composition in a permeable patch that is attachable to a plastic bag prior to the step of applying the composition to the treatment area.

10. The method of claim 9, wherein the permeable patch is formed by extruding the composition in plastic.

11. The method of claim 1, further comprising the step of placing the composition in a permeable sachet prior to the step of applying the composition to the treatment area.

12. The method of claim 1, wherein the step of applying the composition to the treatment area is characterized by distributing the composition over produce.

13. The method of claim 12, wherein the release of chlorine dioxide is accelerated by contacting the produce with moisture.

14. A method of preparing a product usable as a disinfectant and deodorizer, the method comprising impregnating amorphous calcium silicate with a chlorite salt and compressing the mixture into a tablet.

15. The method of claim 14, wherein the chlorite salt is sodium chlorite.

16. The method of claim 14, wherein a desiccant is added to the amorphous calcium silicate to control the release profile of the chlorine dioxide.

17. The product of claim 14, wherein the product further comprises an activator.

18. The product of claim 17, wherein the activator includes a GRAS acid.

19. A product usable as a disinfectant and deodorizer, the product comprising a mixture of amorphous calcium silicate impregnated with a chlorite salt, wherein the mixture is packaged in a permeable sachet.

20. The product of claim 19, wherein the chlorite salt is sodium chlorite.

21. The method of claim 19, wherein a desiccant is added to the amorphous calcium silicate to control the release profile of the chlorine dioxide.

22. The product of claim 19, wherein the product further comprises an activator.

23. The product of claim 19, wherein the activator includes a GRAS acid.

24. A product usable as a disinfectant and deodorizer, the product comprising a mixture of amorphous calcium silicate impregnated with a chlorite salt, wherein the mixture is packaged in a patch attached to a plastic bag.

25. The product of claim 24, wherein the chlorite salt is sodium chlorite.

26. The method of claim 24, wherein a desiccant is added to the amorphous calcium silicate to control the release profile of the chlorine dioxide.

27. The product of claim 24, wherein the product further comprises an activator.

28. The product of claim 27, wherein the activator includes a GRAS acid.

29. The method of claim 24, wherein the patch is formed by extruding the reactant and activator in plastic.

30. A method for reducing spoilage of produce, the method comprising:
applying a reactant to the produce, wherein the reactant includes amorphous calcium silicate impregnated with a chlorite salt; and
stimulating the release of chlorine dioxide by contacting the reactant with an activator, wherein the activator includes an acid.

31. The method of claim 30, wherein chlorite salt is sodium chlorite.

32. The method of claim 30, wherein the acid is a GRAS acid.

33. A method for reducing spoilage of produce, the method comprising:
applying an activator to the produce, wherein the activator includes amorphous calcium silicate impregnated with an acid; and
applying a chlorite salt to stimulate the release of chlorine dioxide.

34. The method of claim 33, wherein chlorite salt is sodium chlorite.

35. The method of claim 33, wherein the acid is a GRAS acid.

36. A method for producing chlorine dioxide in accordance with a step-function release profile, the method comprising:
mixing amorphous silicate with a chlorite salt to produce a reactant;
combining the reactant with an activator to form a composition;
adding a desiccant to the composition to control the release profile of the chlorine dioxide; and
exposing the composition with desiccant to moisture.

37. The method of claim 36, wherein the amorphous silicate is amorphous calcium silicate.

38. The method of claim 36, wherein the amorphous silicate is amorphous magnesium silicate.

39. The method of claim 36, wherein the amorphous silicate is expanded amorphous aluminum silicate.

40. The method of claim 36, wherein the chlorite salt is sodium chlorite.

41. The method of claim 36, wherein the activator comprises amorphous calcium silicate impregnated.

42. The method of claim 41, wherein the acid is a GRAS acid.
